(19) United States
(12) Patent Application Publication
(10) Pub. No.: US 2006/0159741 A1
(43) Pub. Date: Jul. 20, 2006

(54) Pharmaceutical compositions comprising amoxicillin and clavulanic acid

(76) Inventors: Janez Kerc, Ljubljana (SI); Mateja Salobir, Ljubljana (SI)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/536,543

(22) PCT Filed: Nov. 25, 2003
(86) PCT No.: PCT/SI03/00043

§ 371 Date: Jun. 28, 2005

(30) Foreign Application Priority Data
Nov. 26, 2002 (SI) P-200200282
Sep. 24, 2003 (SI) P-200300247

(51) Int. Cl. A61K 9/48; A61K 9/26; B27N 3/02

(52) U.S. Cl. 424/451; 424/469; 264/109

(57) ABSTRACT

The present invention relates to the formed particles comprising amoxicillin and clavulanic acid, the particles being obtained wet granulation. The invention also relates to the procedure for the preparation of these particles and to the pharmaceutical compositions comprising them.

The present invention belongs to the field of pharmaceutical technology and relates to pharmaceutical compositions comprising a combination of the beta-lactam antibiotic amoxicillin and beta lactamase inhibitor clavulanic acid.

The present invention relates to formed particles comprising amoxicillin and clavulanic acid, the particles being obtained by wet granulation. The object of the invention is also a process for the preparation of these particles and pharmaceutical compositions comprising them.

The medicaments which comprise the combination of amoxicillin and clavulanic acid are available on the market in different forms: as conventional immediate-release tablets, powder for reconstitution into water suspension, sachets, chewing tablets, multilayer tablets. The formulations with the ratio of amoxicillin to clavulanic acid 2:1, 4:1, 7:1, 8:1, 14:1 or 16:1 are known. There is a constant need for improved stable farmaceutical compositions comprising amoxycillin and clavulanic acid.

The tablets containing amoxicillin and clavulanic acid are usually prepared first by dry granulation (slugging, compacting) of the active substance and a portion of the excipients. The resulting granulate is then mixed with the remainder of the excipients and the mixture is compressed into tablets. The processes for the preparation of these tablets are described, for example, in patent applications WO 92/19227, WO 95/28927 and WO 98/35672.

Patent application WO 01/62231 discloses the pharmaceutical compositions which comprise at least four different types of pellets. Some of them comprise amoxicillin, the others clavulanic acid. Pellets containing both active drugs are not described. Because of different release rates of the active substances from the pellets, the preparation of the formulations having different release profiles is possible.

WO 95/25516 describes the granules which, in addition to amoxicillin and and/or clavulanic acid, comprise one or more surfactants, for example, sodium lauryl sulfate. Sodium lauryl sulfate is used as spheronizing agent.

### DESCRIPTION OF THE INVENTION

It is an object of the invention to provide new formed particles comprising amoxicillin and clavulanic acid, which are suitable for the preparation of improved, stable pharmaceutical compositions of amoxicillin and clavulanic acid.

The particles of the invention are obtained by wet granulation method. For granulation pharmaceutically acceptable organic solvents or mixtures thereof, or binder dispersion in an organic solvent are used. Preferred solvents include acetone, ethanol and acetonitrile, in particular acetone. The particles may be spherical or irregular in shape.

Amoxicillin may be in the form of amoxicillin trihydrate or in the form of sodium crystalline amoxicillin. Clavulanic acid may be in the form of a salt such as potassium clavulanate. The ratio of amoxicillin and clavulanic acid is preferably from 1:1 to 30:1, especially suitable are the ratios 2:1, 4:1, 7:1, 8:1, 12:1, 14:1, 16:1 and 20:1.

The particles of the present invention may comprise, in addition to the active substances, also excipients such as fillers, binders, disintegrants, glidants and lubricants. Suitable fillers are microcrystalline cellulose, powdered cellulose, lactose, starch, pregelatinized starch, sucrose, glucose, mannitol, sorbitol, calcium phosphate, calcium hydrogen phosphate, aluminium silicate, sodium chloride, potassium chloride, calcium carbonate, calcium sulfate, dextrates, dextrin, maltodextrin, glycerol palmitostearate, hydrogenated vegetable oil, kaolin, magnesium carbonate, magnesium oxide, polymethacrylates, talc and others, preferably microcrystalline cellulose and lactose. Suitable binders are starch, pregelatinized starch, gelatine, sodium carboxymethylcellulose, polyvinylpyrrolidone, alginic acid, sodium alginate, acacia, carbomer, dextrin, ethylcellulose, guar gum, hydrogenated vegetable oil, methylcellulose, hydroxyethyl cellulose, hydroxypropyl cellulose, hydroxypropyl methylcellulose, glucose syrup, magnesium aluminium silicate, maltodextrin, polymethacrylates, zein, preferably hydroxypropyl cellulose, hydroxypropyl methylcellulose and polyvinylpyrrolidone. Suitable disintegrants are starch, pregelatinized starch, sodium starch glycolate, sodium carboxymethylcellulose, cross-linked sodium carboxymethylcellulose, calcium carboxymethylcellulose, methylcellulose, microcrystalline cellulose, powdered cellulose, potassium polacrilinin, cross-linked polyvinylpyrrolidone, alginic acid, sodium alginate, colloidal silicon dioxide, guar gum, magnesium aluminium silicate and others, preferably sodium starch glycolate, cross-linked sodium carboxymethylcellulose and cross-linked polyvinylpyrrolidone. Suitable glidants are magnesium stearate, calcium stearate, aluminium stearate, stearic acid, palmitic acid, cetanol, stearol, polyethylene glycol of different molecular weights, magnesium trisilicate, calcium phosphate, colloidal silicon dioxide, talc, powdered cellulose, starch and others, preferably colloidal silicon dioxide. Suitable lubricants are steraic acid, calcium, magnesium, zinc or aluminium stearate, siliconized talc, glycerol monostearate, glycerol palmitostearate, hydrogenated castor oil, hydrogenated vegetable oil, mineral oil, light mineral oil, polyethylene glycol, sodium benzoate, sodium lauryl sulfate, sodium stearyl fumarate, talc and others. Preferred lubricants are magnesium and calcium stearate, and stearic acid.

The particles of the present invention may also comprise the excipients that enhance the absorption of drugs from gastrointestinal tract. Suitable absorption enhancers for amoxicillin may be selected from surface active agents, fatty acids, middle chain glycerides, steroide detergents (salts of bile salts), acyl carnitine and alcanoloil choline (esters of carnitine and choline and fatty acids with middle chain and long chain), N-acyl derivatrives of alpha-amino acids and N-acyl derivatives of non-alpha-amino acids, chitosanes and other mucoadhesive polymers. Especially suitable absorption enhancers are sodium deoxycholate, sodium taurocholate, polisorbate 80, sodium lauryl sulfate, sodium dodecylsulfate, octanoic acid, sodium docusate, sodium laurate, glyceride monolaurate, stearic acid, palmitinic acid, palmitooleinic acid, glycerilmonooleate, sodium taurocholate, ethylenediaminetetraacetic acid, sodium edentate, sodium citrate, β-cyclodextrine and sodium salicylate. Preferred absorption enhancers are sodium deoxycholate, sodium docusate and sodium lauryl sulfate.

The particles of the present invention may also comprise the excipients which control the drug release. They may be different polymers such as methylcellulose, ethylcellulose, hydroxyethyl cellulose, hydroxypropyl methylcellulose, hydroxypropyl cellulose, sodium carboxymethylcellulose, hydroxypropyl methylcellulose phthalate, polyethylene glycols of different molecular weights, different derivatives of acrylic and methacrylic acid, xanthan gum, alginic acid, sodium alginate, polyvinylpyrrolidone, polyethylene oxides, or nonpolymer substances such as, e.g. hydrogenated vegetable oil, hydrogenated castor oil, glycerol monostearate, glycerol palmitostearate and others.

The formed particles of the present invention have good flow and compressible properties. The proportion of the excipients in the particles is advantageously from 10% by weight to 40% by weight. The particle size ranges from 50 pm to 3000 μm.

Formed particles of the invention may optionally be coated with a release controlling coating or with a protective coating. The coating may be prepared from polymer or nonpolymer substances. Suitable polymers that may be used are hydroxypropyl cellulose, hydroxypropyl methylcellulose, methylcellulose, ethylcellulose, hydroxyethyl cellulose, sodium carboxymethylcellulose, cellulose phthalate acetate, polyvinyl acetate phthalate, hydroxymethyl cellulose phthalate, polyvinyl alcohol, methylhydroxyethyl cellulose, polymers of acrylic and methacrylic acid, maltodextrin and others. Nonpolymer substances that may be used are carnauba wax, cetyl alcohol, sucrose, glucose, shellac and others. The coating may optionally comprise other coating agents conventionally used in coating such as fillers, e.g. talc, lactose, polysaccharides and others, plasticizers, e.g. dibutyl sebacate, triethyl citrate, polyethylene glycol, adipic acid, cocoanut oil, oleic acid and others, colourants, e.g. titanium dioxide, lakes, pigments and others, antioxidants and others.

The object of the present invention are also pharmaceutical compositions comprising the above described formed particles. Coated and/or uncoated particles may be used. They may be filled into sachets or capsules, they may be compressed together with suitable excipients into tablets or they mey be used for reconstitution into suspension. Tablets may be single- or multilayer, dispersible, orodispersible, effervescent, chewing, pastilles. The tablets of the invention are hard and have suitable physical technological properties. By the addition of suitable excipients the release of the active substance from the tablet may be controlled. Excipients to be added to the formed particles for the preparation of the previously stated pharmaceutical compositions may be different fillers, binders, disintegrants, glidants and lubricants. Excipients which enhance the absorption of drugs from gastrointestinal tract may also be added.

Suitable fillers may be microcrystalline cellulose, powdered cellulose, lactose, starch, pregelatinized starch, sucrose, glucose, mannitol, sorbitol, calcium phosphate, calcium hydrogen phosphate, aluminium silicate, sodium chloride, potassium chloride, calcium carbonate, calcium sulfate, dextrates, dextrin, maltodextrin, glycerol palmitostearate, hydrogenated vegetable oil, kaolin, magnesium carbonate, magnesium oxide, polymethacrylates, talc and others, preferably microcrystalline cellulose and lactose. Suitable binders are starch, pregelatinized starch, gelatine, sodium carboxymethylcellulose, polyvinylpyrrolidone, alginic acid, sodium alginate, acacia, carbomer, dextrin, ethylcellulose, guar gum, hydrogenated vegetable oil, methylcellulose, hydroxyethyl cellulose, hydroxypropyl cellulose, hydroxypropyl methylcellulose, glucose syrup, magnesium aluminium silicate, maltodextrin, polymethacrylates, zein, preferably hydroxypropyl cellulose, hydroxypropyl methylcellulose and polyvinylpyrrolidone. Suitable disintegrants are starch, pregelatinized starch, sodium starch glycolate, sodium carboxymethylcellulose, cross-linked sodium carboxymethylcellulose, calcium carboxymethylcellulose, methylcellulose, microcrystalline cellulose, powdered cellulose, potassium polacrilinin, cross-linked polyvinylpyrrolidone, alginic acid, sodium alginate, colloidal silicon dioxide, guar gum, magnesium aluminium silicate and others, preferably sodium starch glycolate, cross-linked sodium carboxymethylcellulose and cross-linked polyvinylpyrrolidone. Suitable glidants are magnesium stearate, calcium stearate, aluminium stearate, stearic acid, palmitic acid, cetanol, stearol, polyethylene glycols of different molecular weights, magnesium trisilicate, calcium phosphate, colloidal silicon dioxide, talc, powdered cellulose, starch and others, preferably, colloidal silicon dioxide. Suitable lubricants are steraic acid, calcium, magnesium, zinc or aluminium stearate, siliconized talc, glycerol monostearate, glycerol palmitostearate, hydrogenated castor oil, hydrogenated vegetable oil, mineral oil, light mineral oil, polyethylene glycol, sodium benzoate, sodium lauryl sulfate, sodium stearyl fumarate and others. Preferred lubricants are calcium or magnesium stearate and stearic acid.

Suitable absorption enhancers for amoxicillin may be selected from surface active agents, fatty acids, middle chain glycerides, steroide detergents (salts of bile salts), acyl camitine and alcanoloil choline (esters of carnitine and choline and fatty acids with middle chain and long chain), N-acyl derivatrives of alpha-amino acids and N-acyl derivatives of non-alpha-amino acids, chitosanes and other mucoadhesive polymers. Especially suitable absorption enhancers are sodium deoxycholate, sodium taurocholate, polisorbate 80, sodium lauryl sulfate, sodium dodecylsulfate, octanoic acid, sodium docusate, sodium laurate, glyceride monolaurate, stearic acid, palmitinic acid, palmitooleinic acid, glycerilmonooleate, sodium taurocholate, ethylenediaminetetraacetic acid, sodium edentate, sodium citrate, β-cyclodextrine and sodium salicylate. Preferred absorption enhancers are sodium deoxycholate, sodium docusate and sodium lauryl sulfate.

Polymer or nonpolymer substances may be used as the substances to control the release of the active substance. Suitable polymers that may be used are hydroxypropyl cellulose, hydroxypropyl methylcellulose, methylcellulose, ethylcellulose, hydroxyethyl cellulose, sodium carboxymethylcellulose, cellulose acetate phthalate, polyvinyl acetate phthalate, hydroxymethyl cellulose phthalate, polyvinyl alcohol, methylhydroxyethyl cellulose, sodium carboxymrethylcellulose, polymers of acrylic and methacrylic acid, matodextrin and others. Nonpolymer substance may be carnauba wax, cetyl alcohol, hydrogenated vegetable oil, hydrogenated castor oil, glycerol monosterate, glycerol palmitostearate and others.

Capsules and tablets may optionally be coated with a coating which may be applied from an aqueous or non-aqueous medium. A coating may control the release or it is only a protective coating. The coating may be prepared from polymer or nonpolymer substances. Suitable polymers that may be used are hydroxypropyl cellulose, hydroxypropyl methylcellulose, methylcellulose, ethylcellulose, hydroxyethylcellulose, sodium carboxymethylcellulose, cellulose acetate phthalate, polyvinyl acetate phthalate, hydroxymethyl cellulose phthalate, polyvinyl alcohol, methylhydroxyethyl cellulose, polymers of acrylic and methacrylic acid, maltodextrin and others. Nonpolymer substances may be carnauba wax, cetyl alcohol, sucrose, glucose, shellac and others.

The coating may also comprise other conventionally used coating agents such as fillers, e.g. talc, lactose, polysaccharides and others, plasticizers, e.g. dibytil sebacate, trietyl citrate, polyethylene glycol and others; colorants, e.g. titanium dioxide, lakes, pigments and others, antioxidants and others.

The formed particles are suitable for the preparation of multiple unit forms such as capsules or tablets with the primary formed particles wherein a multiple unit form disintegrates to individual primary formed particles from which the active substance is released. The formed particles are also suitable for the preparation of multiple unit forms such as sachets and dozers for multiple unit systems and others. Uncoated and/or coated particles may be used.

The compositions of the present invention may preferably contain from 250 to 1500 mg of amoxicillin and the appropriate amount of clavulanic acid. They may be prepared, for example as 250/125, 500/125, 500/62.5, 875/125, 1000/125, 1000/62.5, 400/57, 200/28.5 unit dosage forms.

The release of amoxicillin and clavulanic acid from the pharmaceutical formulations of the present invention can be immediate or modified, controlled, delayed, sustained, extended. The release rate for both active drugs can be the same or different. In the case the release of amoxicillin and clavulanic acid is different, the release of amoxicillin is slower than the release of clavulanic acid. Such release profile may increase the bioavailability of amoxicillin.

The pharmaceutical formulations of the present invention may comprise formed particles with the same composition or formed particles with different composition and different release rate of amoxicillin and clavulanic acid.

The formed particles of the present invention comprising amoxicillin and clavulanic acid may be combined with the similar particles comprising only amoxicillin.

The present invention also relates to the process for the preparation of the formed particles regular and irregular in shape. They are prepared by the wet granulation with organic solvent. For granulation either a solvent or binder dispersion in an organic solvent may be used.

The formed particles irregular in shape are prepared by the procedure including the following key steps:
preparation of the mixture of amoxicillin trihydrate and potassium clavulanate and excipients (with or without a binder)
wet granulation with an organic solvent or wet granulation with a binder dispersion in an organic solvent
drying of particles
grinding or sieving of dry particles
optionally application of a coating

The formed particles spherical in shape (spheronizates, pellets) are prepared by the procedure including the following key steps:
preparation of the mixture of amoxicillin trihydrate and potassium clavulanate and excipients (with or without a binder)
wet granulation with an organic solvent or wet granulation with a binder dispersion in an organic solvent
extrusion of a wet mixture through a screen
spheronization
drying of particles
optionally application of a coating

The most suitable solvents that may be used for granulation are pharmaceutically acceptable organic solvents, not including isopropanol and methylene dichloride.

The most suitable solvents are acetone, ethanol and acetonitrile. Particularly preferred is acetone.

A binder may be dispersed in an organic solvent to obtain a solution or a suspension and this dispersion is used for granulation. Preferable organic solvents for the preparation of a binder dispersion include acetone, ethanol and acetonitrile, particularly prefered is acetone.

Potassium clavulanate is a highly moisture sensitive substance. Therefore, prior to use all excipients are previously dried, or already pre-dried excipients are used, or the formed particles are adequately dried at the end. The production is carried out under dry conditions of relative humidity not exceeding 30%.

The present invention is illustrated but in no way limited by the following examples:

### EXAMPLE 1

Tablets 875/125 mg

| Ingredients | weight | |
|---|---|---|
| Amoxicillin | 875.00 | mg |
| as trihydrate | 1008.00 | mg |
| Clavulanic acid | 125.00 | mg |
| as potassium clavulanate | 149.88 | mg |
| Lactose anhydrous | 154.12 | mg |
| Hydroxypropyl cellulose | 20.00 | mg |
| Microcrystalline cellulose | 14.00 | mg |
| Cross-linked sodium carboxymethylcellulose | 10.00 | mg |
| Magnesium stearate | 5.00 | mg |
| Total | 1361.00 | mg |
| Acetone | 630.00 | mg |

Method of preparation: amoxicillin trihydrate, potassium clavulanate, lactose, microcrystalline cellulose, hydroxypropyl cellulose and cross-linked carboxymethylcellulose were homogeneously mixed and granulated with acetone to produce formed particles. Wet formed particles were dried under vacuum at 40° C. (approximately 3 hours, to loss on drying of 6% (80° C., 20 min). To the dried formed particles magnesium stearate was added and the mixture was homogeneously mixed. Tablets of the adequate weight and suitable physical and technological properties were compressed on a conventional tablet press.

Stability tests show the tablets are stable.

### EXAMPLE 2

Tablets 875/125 mg

| Ingredients | weight | |
|---|---|---|
| Amoxicillin | 875.00 | mg |
| as trihydrate | 1008.00 | mg |
| Clavulanic acid | 125.00 | mg |
| as potassium clavulanate | 149.88 | mg |
| Lactose anhydrous | 154.12 | mg |
| Polyvinylpyrrolidone | 20.00 | mg |
| Microcrystalline cellulose | 14.00 | mg |
| Cross-linked sodium carboxymethylcellulose | 10.00 | mg |
| Magnesium stearate | 5.00 | mg |
| Total | 1361.00 | mg |
| Acetone | 630.00 | mg |

Method of preparation: amoxicillin trihydrate, potassium clavulanate, lactose, microcrystalline cellulose, polyvinylpyrrolidone and cross-linked carboxymethylcellulose were homogeneously mixed and granulated with acetone to produce formed particles. Wet formed particles were dried under vacuum at 40° C. (approximately 3 hours, to loss on drying of 6% (80° C., 20 min). To the dried formed particles magnesium stearate was added and the mixture was homogeneously mixed. Tablets of adequate weight and suitable physical and technological properties were compressed on a conventional tablet press.

Stability tests show the tablets are stable.

### EXAMPLE 3

Tablets 875/125 mg

| Ingredients | weight | |
|---|---|---|
| Amoxicillin | 875.00 | mg |
| as trihydrate | 1008.00 | mg |
| Clavulanic acid | 125.00 | mg |
| as potassium clavulanate | 149.88 | mg |
| Lactose anhydrous | 14.00 | mg |
| Polyvinylpyrrolidone | 20.00 | mg |
| Microcrystalline cellulose | 154.12 | mg |
| Cross-linked sodium carboxymethylcellulose | 10.00 | mg |
| Magnesium stearate | 5.00 | mg |
| Total | 1361.00 | mg |
| Acetone | 630.00 | mg |

Method of preparation: amoxicillin trihydrate, potassium clavulanate, lactose, microcrystalline cellulose, polyvinylpyrrolidone and cross-linked carboxymethylcellulose were homogeneously mixed and granulated with acetone to produce formed particles. Wet formed particles were dried under vacuum at 40° C. (approximately 3 hours to loss on drying of 6% (80° C., 20 min). To the dried formed particles magnesium stearate was added and the mixture was homogeneously mixed. Tablets of adequate weight and suitable physical and technological properties were compressed on a conventional tablet press.

Stability tests show the tablets are stable.

### EXAMPLE 4

Pellets 875/125 mg

| Ingredients | weight | |
|---|---|---|
| Amoxicillin | 875.00 | mg |
| as trihydrate | 1011.56 | mg |
| Clavulanic acid | 125.00 | mg |
| as potassium clavulanate | 151.33 | mg |
| Lactose anhydrous | 149.11 | mg |
| Hydroxypropyl cellulose | 20.00 | mg |
| Microcrystalline cellulose | 14.00 | mg |
| Cross-linked sodium carboxymethylcellulose | 10.00 | mg |
| Total | 1356.00 | mg |
| Acetone | 730.00 | mg |

Method of preparation: amoxicillin trihydrate, potassium clavulanate, lactose, microcrystalline cellulose, hydroxypropylcellulose and cross-linked carboxymethylcellulose were homogeneously mixed and granulated with acetone. The wet mass was extruded through a screen, opening size 0.8 mm, to produce extrudates. The wet extrudates were then spheronized in a spheronization apparatus (spheronizer) to produce spherical-shaped formed particles. These spherical-shaped formed particles were dried under vacuum at 40° C. (approximately 3 hours, to loss on drying of 6% (80° C., 20 min).

### EXAMPLE 5

Pellets 875/125 mg

| Ingredients | weight | |
|---|---|---|
| Amoxicillin | 875.00 | mg |
| as trihydrate DC | 1019.80 | mg |
| Clavulanic acid | 125.00 | mg |
| as potassium clavulanate | 150.00 | mg |
| Lactose anhydrous | 142.20 | mg |
| Hydroxypropyl cellulose | 20.00 | mg |
| Polyvinylpyrrolidone | 20.00 | mg |
| Microcrystalline cellulose | 14.00 | mg |
| Cross-linked sodium carboxymethylcellulose | 10.00 | mg |
| Total | 1376.00 | mg |
| Acetone | 630.00 | mg |

Method of preparation: amoxicillin trihydrate, potassium clavulanate, lactose, microcrystalline cellulose, hydroxypropyl cellulose, polyvinylpyrrolidone and cross-linked carboxymethylcellulose were homogeneously mixed and granulated with acetone. The wet mass was extruded through a screen, opening size 1.5 mm, to produce extrudates. The wet extrudates were then spheronized in a spheronization apparatus (spheronizer) to produce regular spherical-shaped formed particles. These spherical-shaped formed particles were dried under vacuum at 40° C. (approximately 3 hours, to loss on drying of 6% (80° C., 20 min).

Stability tests show the tablets are stable.

### EXAMPLE 6

Pellets 875/125 mg

| Ingredients | weight | |
|---|---|---|
| Amoxicillin | 875.00 | mg |
| as trihydrate DC | 1019.80 | mg |
| Clavulanic acid | 125.00 | mg |
| as potassium clavulanate | 150.00 | mg |
| Lactose anhydrous | 142.20 | mg |
| Hydroxypropyl cellulose | 20.00 | mg |
| Polyvinylpyrrolidone | 20.00 | mg |
| Microcrystalline cellulose | 14.00 | mg |
| Cross-linked sodium carboxymethylcellulose | 10.00 | mg |
| Total | 1376.00 | mg |
| Ethanol | 730.00 | mg |

Procedure:

Method of preparation: amoxicillin trihydrate, potassium clavulanate, lactose, microcrystalline cellulose, hydroxypropylcellulose, polyvinylpyrrolidone and cross-linked carboxymethylcellulose were homogeneously mixed and granulated with ethanol. The wet mass was extruded through a screen, opening size 1.5 mm, to produce extrudates. The wet extrudates were then spheronized in a spheronization apparatus (spheronizer) to produce regular spherical-shaped formed particles. These spherical-shaped formed particles were dried under vacuum at 40° C. (approximately 3 hours, to loss on drying of 6% (80° C., 20 min).

1. Formed particles comprising amoxicillin and clavulanic acid, wherein the particles are obtained by wet granulation.

2. The formed particles according to claim 1, wherein amoxicillin is present in the form of amoxicillin trihydrate and clavulanic acid is present in the form of potassium clavulanate.

3. The formed particles according to claim 1, wherein the ratio of amoxicillin to clavulanic acid is from 1:1 to 30:1.

4. The formed particles according to claims 1 and 3, wherein the ratio of amoxicillin to clavulanic acid is 4:1, 7:1, 8:1, 12:1, 16:1, 20:1.

5. The formed particles according to claim 1, further comprising excipients selected from fillers, binders, disintegrants, glidants, lubricants.

6. The formed particles of claim 1, wherein the solvent used in the wet granulation is an organic solvent.

7. The formed particles according to claims 1 and 6, wherein the organic solvent is acetone.

8. The formed particles according to claim 1, wherein a binder dispersion in an organic solvent is used in wet granulation.

9. The formed particles according to claim 1 and 8, wherein the binder is hydroxypropyl cellulose and/or polyvinylpyrrolidone.

10. The formed particles according to claim 1, which further comprise excipients that increase the absorption of amoxicillin.

11. The formed particles according to claims 1 and 10, wherein the excipients that increase the absorption of amoxicillin are selected from the group of sodium deoxycholate, sodium docusate and sodium lauryl sulfate.

12. The formed particles according to claim 1, which are irregular in shape or spherical.

13. The formed particles according to claim 1, which are coated or uncoated.

14. A pharmaceutical composition comprising the formed particles according to claim 1.

15. The pharmaceutical composition according to claim 14, wherein the formed particles are irregular-shaped.

16. The pharmaceutical composition according to claim 14, wherein the formed particles are spherical.

17. The pharmaceutical composition according to claim 14 in the form of a multiple unit formulation.

18. The pharmaceutical composition according to claim 14 in the form of a tablet, capsule or sachet.

19. The pharmaceutical composition according to claim 14, which comprise excipients that increased the absorption of amoxicillin.

20. The pharmaceutical composition according to claim 14 and 19, wherein the excipients that increase the absorption of amoxicillin are selected from the group of sodium deoxycholate, sodium docusate and sodium lauryl sulfate.

21. The pharmaceutical composition according to claim 14, which has a coating.

22. A process for the preparation of the formed particles according to claim 1 comprising the following steps:
preparation of the mixture of amoxicillin trihydrate and potassium clavulanate and excipients (with or without a binder),
wet granulation with an organic solvent or wet granulation with a binder dispersion in an organic solvent,
drying of particles,
grinding or sieving of dry particles,
optionally application of a coating

23. A process for the preparation of the formed particles according to claim 1 comprising the following steps:
preparation of the mixture of amoxicillin trihydrate and potassium clavulanate and excipients (with or without a binder),
wet granulation with an organic solvent or wet granulation with a binder dispersion in an organic solvent,
extrusion of a wet mixture through a screen,
spheronization,
drying of particles,
optionally application of a coating

24. The procedure for the preparation of the formed particles according to claims 22 and 23, wherein the organic solvent is acetone.
